(19) United States
(12) Patent Application Publication
(10) Pub. No.: US 2026/0283543 A1
(43) Pub. Date: Sep. 24, 2026

(54) SLEEPING SYSTEM COMPRISING A MATTRESS

(71) Applicant: Vincenzo BUONINFANTE, Montecorvino Rovella (SA) (IT)

(72) Inventor: Vincenzo BUONINFANTE, Montecorvino Rovella (SA) (IT)

(21) Appl. No.: 19/474,458

(22) PCT Filed: Apr. 10, 2024
(86) PCT No.: PCT/IB2024/053495

§ 371 Date: Oct. 10, 2025

(30) Foreign Application Priority Data
Apr. 13, 2023 (IT) 102023000007083

(51) Int. Cl. A61B 5/00; A61B 5/024; A61B 5/28

(52) U.S. Cl. CPC A61B 5/4812; A61B 5/024; A61B 5/28; A61B 5/4809; A61B 5/6892; A61B 2560/0242; A61B 2560/0462

(57) ABSTRACT

A sleeping system comprises a mattress and a set of sensors for detecting and monitoring the mattress user's vital signs and sleeping parameters.

The present invention relates to a sleeping system comprising a mattress and a set of sensors for detecting and monitoring the mattress user's vital signs and sleeping parameters.

To monitor people's health, it is becoming increasingly important to detect their sleep quality.

Sleep quality detection systems are increasingly being used for this purpose. Such systems usually involve wristbands that only detect certain sleep parameters.

Mats inserted under the mattress and comprising a set of sensors are also used according to the known technique.

The detection carried out is, however, limited because the distance of the sensors from the person lying on the mattress is considerable.

The known technique also provides for complete bed systems, comprising a bed base, mattress and footboard at the base, to which sensors are inserted, but which are not integrated and connected to each other.

It is an object of the present invention to provide a sleeping system that overcomes the problems of the known technique.

It is a further scope of the invention to provide a sleeping system that allows vital and sleep parameters to be reliably monitored.

A further purpose of the invention is to provide a sleeping system that provides values relating to vital parameters and sleep parameters in an organised and easily visible manner.

These and other purposes are achieved by a sleeping system for monitoring a person's vital and sleep parameters according to the appended claims.

Further features and details may be better understood from the following description, given by way of non-limiting example.

A sleeping system according to the invention comprises:
a mattress;
a group of sensors integrated within the mattress;
an electronic control unit connected to the sensors and located in a special pocket in the mattress.

The mattress included in the sleeping system can be of two types:
a hybrid mattress with pocketed springs, comprising an internal structure with steel pocketed springs enclosed in non-woven fabric (TNT) bags and polyurethane foam, additional layers of polyurethane foam, hypoallergenic fibres and an outer cover
polyurethane foam mattress, comprising a supporting polyurethane foam sheet, additional layers of polyurethane foam, non-allergenic fibres and outer covering.

Once integration with the sensors is complete, the mattress is bagged, pressed until its thickness is reduced to a few centimetres, and finally vacuum-rolled so that it is ready for delivery to the end user.

The group of sensors inside the mattress includes, first of all, pressure sensors evenly distributed over the entire surface of the mattress facing the user and are designed to detect the pressure exerted on the mattress and return the input to the electronic control unit.

The sensors are connected to each other via cables and are also connected to an output cable connected, via a hole drilled in the mattress, to the electronic control unit.

Thanks to the presence of these pressure sensors and the measurement made by them over a period of time, the sleeping system monitors and tracks sleep-related parameters such as
the time spent in bed
the time of falling asleep
the duration of rest;
the position assumed during sleep and during rest;
the stages of sleep and its interruptions.

The group of sensors also includes a heart rate sensor, an electrocardiogramsor, and at least one audio sensor integrated into the mattress.

Thanks to these sensors, the sleeping system can detect vital parameters such as:
heart rate
electrocardiogram (ECG);
respiratory frequency;
snoring.

In addition, the sensor group also includes a temperature sensor inside the mattress, a temperature sensor outside the mattress, and an audio sensor outside the mattress.

Thanks to these sensors, the sleeping system can also measure environmental parameters such as:
the temperature inside the mattress
the outside temperature;
the external noise.

The electronic control unit is placed in a special pocket in the mattress.

The electronic control unit interacts with the sensor group by means of a cable connected to each sensor in the sensor group, which is outgoing from the mattress.

The electronic control unit collects the output data from each of the sensors in the sensor group and processes them on a time basis, also according to the initially defined settings.

The sleeping system also includes a software application for electronic devices such as smartphones, tablets and personal computers.

The electronic control unit connects via Bluetooth or other connection system with the electronic device on which the software application is running.

The data processed by the electronic control unit can then be sent to the electronic device, and the software application can show the user the data it has collected and processed.

The electronic application can thus show the user the data in real time, can illustrate reports and periodic trends on the measured parameters.

Optionally and non-bindingly, the user can also use T-shirts or wristbands equipped with sensors and connected to the electronic control unit.

The sleeping system according to the invention can be used in the following way:
the user lies down on the mattress;
the sensor unit detects the presence of the user's body on the mattress and initiates a measurement of pressure and body position parameters as well as vital parameters such as heart rate and respiratory rate.
The sensors transmit the detected data to the electronic control unit;
the electronic control unit also processes the data received on a time scale;
the electronic control unit sends the processed data to one or more electron devices on which the software application is running;
the software application carries out any further processing of the data and displays the processed data to the user via a graphic interface and in real time, also showing reports of aggregated data and periodic trends.

The presence of sensors of various types also allows the detection and display of environmental parameters such as temperature in the mattress, external temperature and external noise.

Variants and methods of implementation other than those illustrated above are possible and are to be considered within the scope of protection defined by the attached claims.

For example, each sensor may comprise an autonomous system for direct connection to the electronic device on which the software application is intended.

1.-7. (canceled)

8. Sleeping system suitable for monitoring a user's vital signs and sleep parameters, comprising:
a mattress on which the user can lie;
a group of sensors comprising at least one sensor integrated inside the mattress and at least one sensor placed outside the mattress;
an electronic control unit connected to the sensors and placed in a special pocket made in the mattress.

9. The sleeping system according to claim 8, further comprising a software application running on an electronic device such as a smartphone, tablet, personal computer, the electronic device being connectable to the electronic control unit.

10. The sleeping system according to claim 8, further comprising a garment or accessory wearable by the user and connected to the electronic control unit.

11. The sleeping system according to claim 9, further comprising a garment or accessory wearable by the user and connected to the electronic control unit.

12. The sleeping system according to claim 8, wherein the group of sensors comprises at least one pressure sensor disposed close to the contact surface of the mattress with the user.

13. The sleeping system according to claim 8, further comprising a garment or accessory wearable by the user and connected to the electronic control unit, wherein the group of sensors comprises at least one pressure sensor disposed close to the contact surface of the mattress with the user.

14. The sleeping system according to claim 8, wherein the group of sensors includes at least one of the following sensors:
a heart rate sensor;
an electrocardiogramaensor;
an audio sensor built into the mattress;
a temperature sensor arranged inside the mattress;
a temperature sensor arranged outside the mattress;
an audio sensor arranged outside the mattress.

15. The sleeping system according to claim 8, further comprising a garment or accessory wearable by the user and connected to the electronic control unit, wherein the group of sensors includes at least one of the following sensors:
a heart rate sensor;
an electrocardiogramaensor;
an audio sensor built into the mattress;
a temperature sensor arranged inside the mattress;
a temperature sensor arranged outside the mattress;
an audio sensor arranged outside the mattress.

16. The sleeping system according to claim 8, wherein the mattress is a hybrid pocketed spring mattress comprising an inner structure with steel pocketed springs enclosed in non-woven fabric (TNT) bags and polyurethane foam, additional layers of polyurethane foam, hypoallergenic fibers, and outer covering.

17. The sleeping system according to claim 8, wherein the mattress is a polyurethane foam mattress comprising a polyurethane foam carrier sheet, additional layers of polyurethane foam, hypoallergenic fibers, and outer cover.
